(19) United States
(12) Patent Application Publication
(10) Pub. No.: US 2025/0270148 A1
(43) Pub. Date: Aug. 28, 2025

(54) METHOD AND SYSTEM FOR TREATING ANIMAL PRODUCTS

(71) Applicant: COMMISSARIAT A L'ENERGIE ATOMIQUE ET AUX ENERGIES ALTERNATIVES, Paris (FR)

(72) Inventor: Jean-Philippe DESLYS, Fontenay-aux-Roses (FR)

(73) Assignee: COMMISSARIAT A L'ENERGIE ATOMIQUE ET AUX ENERGIES ALTERNATIVES, Paris (FR)

(21) Appl. No.: 18/266,935

(22) PCT Filed: Dec. 14, 2021
(86) PCT No.: PCT/EP2021/085595

§ 371 Date: Jun. 13, 2023

(30) Foreign Application Priority Data
Dec. 15, 2020 (FR) FR2013275

(51) Int. Cl. C05F 17/05; A01G 9/14; C05F 3/06; C05F 17/20

(52) U.S. Cl. CPC C05F 17/05; A01G 9/14; C05F 3/06; C05F 17/20

(57) ABSTRACT

A method for processing animal products, in particular products from poultry farming, includes: a) chemically treating by placing collected waste in contact with an ammonia-based buffer with a pH of at least 8; b) heating the material resulting from step a) in a humid medium to a temperature of at least 70° C.; c) separating the organic matter and inorganic matter in the presence of water from the material resulting from step b), then separated collection of (i) the liquid fraction having the inorganic matter, and (ii) the solid fraction having the organic matter; and d) adding the liquid fraction resulting from step c (ii), having the inorganic matter, in a basin filled with water held in a controlled manner at a setpoint temperature ranging from 20° C. to 42° C., preferably from 28° C. to 35° C., in which a protein-rich plant or bacterium is cultivated, in particular microalgae or cyanobacteria, preferably spirulina.

### TECHNICAL FIELD

The present description relates to the field of the treatment of animal products, in particular of products generated by the practice of animal farming, in particular of poultry, according to techniques allowing respect of the environment, compatible with sustainable development.

### PRIOR ART

The development of food industry activities on an industrial scale has seen rapid growth in recent years. In particular, the increase in poultry production, due to increasing demand for poultry meat and egg products, leads to residual waste that gives rise to several difficulties. In fact, one of the major problems facing the poultry sector is the accumulation of this waste on a large scale and thus raises concerns regarding disposal and pollution (Bolan et al. World's Poultry Science Journal, 2010).

The existing methods for treating the organic waste generated, such as carcasses, excrement, feathers, and poultry litter, are traditional methods such as burial, incineration, flaying and composting. These methods cause environmental, biosafety, social, and economic problems (Baba et al. 2017 J Dairy Vet Anim Res).

Moreover, poultry farming generates a large amount of ammonium and carbon dioxide (CO₂) in the atmosphere, which is a serious problem for animal health (irritation of the trachea, ocular lesions, decrease in feed efficiency and mortality) and for the workers present (respiratory problems), as well as for the environment (Pereira et al. Environ Sci Pollut Res Int. 2019).

It is therefore urgent to develop new technologies for treating this waste for sustainable management in environmental and economic respects.

A great many methods for recycling the waste generated by animal farming practice are described in the prior art.

The document Baba et al. (Int. J. Curr. Microbiol. App. Sci (2018)) proposes the use of fermentation processes for waste recycling and processing. Lactic fermentation contributes to decontamination of carcasses and allows subsequent use of the end product as fermented feed, for other animals.

The document Brandelli et al. (Food Research International (2015)) proposes the use of enzymes in order to convert poultry byproducts into useful products. However, these solutions do not allow efficient and sustainable treatment of all the waste generated by this large-scale farming.

Patent application US 2017/0354906 describes a bioreactor system intended for the treatment of solid waste. Patent application US 2016/0264484 describes an integrated system for animal waste treatment comprising a unit for methanation of animal waste, which generates a biogas and a crude digestate. The crude digestate is separated into a solid fraction and a liquid fraction. Then the liquid fraction is treated in reactors containing microalgae or macrophytes, and the solid fraction is treated by vermiculture. The PCT application published under No. WO 2017/101654 describes a system for treating slurry and wastewater comprising a desalination device and a microalgae culture tank. Patent application EP 3 181 524 describes a system for treating raw materials, mainly animal waste, by anaerobic fermentation in a pressurized bioreactor.

There is therefore a need to develop a method for processing animal waste that is more environmentally friendly, and with enhanced compatibility, with a view to sustainable development, and which if applicable is of a moderate cost price.

In particular, it would be desirable to design new methods allowing the use of organic and mineral waste in order to obtain products that can be recycled, in particular in the form of products for human or animal nutrition.

The need for methods of this type, which would allow a low supply of external energy, or which have a neutral or even negative carbon balance, is considerable in regions that are structurally unsuited to intensive farming, in particular owing to their aridity, such as certain regions of the Middle East and Africa.

There is a need for new methods allowing conversion of animal waste to food in a hostile, in particular arid, environment.

### SUMMARY OF THE INVENTION

The present description relates to a method for treating animal products, in particular products from poultry farming, comprising the following steps:
a) chemical treatment by putting the collected waste in contact with an ammonia-based buffer at pH of at least 8,
b) thermal treatment of the material obtained in step a), by heating said material in humid conditions at a temperature of at least 70° C.,
c) separating the organic matter and the mineral matter in the presence of water, from the material obtained in step b) and then separate collection (i) of the liquid fraction comprising the mineral matter and (ii) of the solid fraction comprising the organic matter, and
d) adding the liquid fraction obtained in step c (ii), comprising the mineral matter, to a tank filled with water maintained in a controlled manner at a setpoint temperature from 20° C. to 42° C., preferably from 28° C. to 35° C., in which a protein-rich plant or bacterium is cultured, in particular microalgae and cyanobacteria, preferably Spirulina.

In certain embodiments of the method, the tank used in step d):
is contained in an enclosure substantially impervious to the liquid and gaseous fluids in the surroundings, said enclosure comprising a roof at least partially transparent to daylight, said roof being equipped with a plurality of photovoltaic cells, preferably Grätzel photovoltaic cells, and/or
is equipped with a device for stirring the water and recovering the protein-rich plant or bacterium,
is supplied with luminous energy, and/or
is equipped with a device regulating the temperature of the water contained in the tank.

According to certain embodiments of the method, for heating the water contained in the tank the temperature regulating device is in fluidic communication with an outlet pipe of the hot water from apparatus for desalination by evaporation/concentration.

According to certain embodiments of the method, the heat exchanger comprises a geothermal exchanger for cooling the water.

According to certain embodiments of the method, said tank is provided with a controllable device for water supply, said water supply device being in fluidic communication with a salt water outlet pipe of a device for desalination of water by evaporation/concentration.

According to certain embodiments of the method, step c) is followed by a step c1) of digestion of the material obtained in step c) by putting said material in contact with (i) insect larvae, preferably insect larvae of the species Hermetia illucens and/or (ii) an edible lignivorous fungus.

The present description also relates to a system for treating animal products, said system comprising:
a reactor for chemical and thermal treatment of said animal products,
an extrusion device equipped with a controlled heating system, which may be in fluidic communication with said reactor, the outlet of the extrusion device being equipped with a liquid/solid separator,
a closed enclosure comprising a tank surmounted by a roof consisting of a material substantially transparent to light, said tank being fillable with water and intended for culture of a plant or a bacterium, in particular a protein-rich plant or bacterium such as protein-rich microalgae or protein-rich cyanobacteria,
a system for water desalination by evaporation/concentration comprising (i) a salt water supply pipe, a pipe for discharge of the water that has not been desalinated and an outlet pipe for desalinated water, it being specified that:
optionally, said reactor is in fluidic communication with the extrusion device,
said tank is provided with a controllable device for water supply, said water supply device being in fluidic communication with a salt water outlet pipe from the desalination system.

In certain embodiments, the system further comprises a bioclimatic greenhouse (passive solar) device comprising:
a closed enclosure comprising a floor and a roof, the floor being located below ground level,
said roof being substantially transparent to light, on the wall of which there is a plurality of photovoltaic cells, preferably Grätzel cells,
said enclosure being provided with a device for dehumidifying the internal atmosphere of the enclosure,
said enclosure being temperature-controlled,
a substrate suitable for growing plants being disposed on the surface of said floor, said substrate consisting, at least partly, of the solid fraction of the product from animal waste treatment, in, successively, the reactor and then the extrusion device.

In certain embodiments of the system, the temperature regulating device of the enclosure of the bioclimatic greenhouse device comprises a heat exchanger in fluidic communication with the water desalination system.

In certain embodiments, the system further comprises an agroforestry arrangement comprising:
an area planted with shade-generating trees arranged in rows suitably spaced to provide a tunnel greenhouse, or a plurality of tunnel greenhouses, between two rows of said trees,
a tunnel greenhouse, or a plurality of tunnel greenhouses, arranged between two rows of trees, and
a system for irrigation of the trees, and if applicable a system for irrigation of the plants that may be grown in the tunnel greenhouse(s), said irrigation system being in fluidic communication with the desalinated water outlet of the system for water desalination by evaporation/condensation.

### BRIEF DESCRIPTION OF THE DRAWINGS

FIG. 1 shows a general schematic of the system for treating animal products.

### DETAILED DESCRIPTION

The present description relates to a method and a system for treating animal products, in particular for treating products generated by poultry farming practice, the method and the system being designed for (i) giving a reduced consumption of water and energy, (ii) allowing optimal use of the organic and mineral matter contained in these animal products, with a view to animal farming practice compatible with sustainable development, respecting the environment.

In the sense of the present description, an “animal product” comprises, or consists of, all or part of an animal carcass as well as substances secreted or excreted by an animal.

Preferably, the animal product is an animal byproduct.

“Animal byproduct” means an animal product, which may or may not be suitable for human consumption, but is not intended for human consumption, either to comply with legislation or for commercial reasons. As examples, an animal byproduct in the sense of the description comprises, or consists of, muscle, internal organs, skin, hooves, horns, feathers, bones, shells, greaves, blood, milk, ovules, embryos, semen, biomass, slurry, methanation residues or mixtures thereof.

The animals from which the animal products or byproducts are derived, in the sense of the description, may be animals kept for profit, such as bovines, sheep, goats, pigs, rabbits and hares, birds or fishes, or other animals, such as horses, pets, arthropods, in particular insects and crustacea, reptiles or mollusks. The animals include quite especially birds kept for profit, in particular poultry such as geese, turkeys, ducks, hens and chicken, guinea fowl, capons, quails, pheasant and pigeons.

### Method According to the Present Description

The present description relates to a method for treating animal products, in particular products from poultry farming, comprising the following steps:
a) chemical treatment by putting the collected waste in contact with an ammonia-based buffer at pH of at least 8,
b) thermal treatment of the material obtained in step a), by heating said material in humid conditions at a temperature of at least 70° C.,
c) separating the organic matter and the mineral matter in the presence of water from the material obtained in step b) and then separate collection (i) of the liquid fraction comprising the mineral matter and (ii) of the solid fraction comprising the organic matter, and
d) adding the liquid fraction obtained in step c (ii), comprising the mineral matter, to a tank filled with water maintained in a controlled manner at a setpoint temperature from 20° C. to 42° C., preferably from 28° C. to 35° C., in which a protein-rich plant or bacterium is cultured, in particular microalgae or cyanobacteria, preferably Spirulina.

As is shown in the example, the method according to the present description allows destruction of the pathogens that may be contained in the animal starting products. It is shown in particular that this method may allow total destruction of the pathogens contained in very heavily contaminated animal starting products. Thus, the results show that the method according to the present description results in the total destruction of pathogenic prions contained in the animal starting products, including when the level of contamination of the animal starting products is greater than 10⁸ LD₅₀/g.

Preferably, according to the method for treating animal products, the plants or the bacteria consist of photosynthetic plants or photosynthetic bacteria, of a known type.

In the sense of the present description, the cyanobacteria comprise mainly, or even exclusively, cyanobacteria that are nontoxic to humans and animals.

In certain embodiments, the animal product is ground prior to application of the steps of the above method, in order to obtain a granular product with an average particle size, or granulometry, advantageously of at most 20 mm, preferably at most 10 mm, and quite preferably at most 5 mm.

### Step a): Chemical Treatment

In step a) of the method, the animal product is submitted to a chemical treatment with an ammonia-based buffer at pH of at least 8.

Advantageously, the animal product is in the form of small pieces, so as to optimize its treatment in the various steps of the method. Typically, the animal product to be treated is in the form of pieces whose largest dimension varies from some millimeters to some centimeters.

Preferably, aqueous ammonia solution is used.

The amount of ammonia solution relative to the weight of animal product may be determined optimally by a person skilled in the art, in particular as a function of the order of magnitude of the water content of the animal product to be treated.

As an illustration, 5 mL of aqueous ammonia solution at 0.67% w/w may be used for 15 grams of animal product to be treated.

The chemical treatment with aqueous ammonia solution reduces the water loss of the animal product during the next step of thermal treatment.

The chemical treatment makes it possible to reduce substantially any contamination of the animal product with pathogens, in particular pathogenic viruses, bacteria and fungi.

In certain embodiments, the aqueous ammonia solution also comprises citrate. In these embodiments, the chemical treatment step allows enhanced reduction of any contamination with pathogens, including unconventional pathogens such as pathogenic proteins of the prion type.

According to another advantage, step a) of chemical treatment avoids the phenomenon of shrinkage of the animal product that occurs during a thermal treatment. Thus, owing to the chemical treatment carried out in step a), the animal product does not shrink when it is then submitted to step b) of thermal treatment, which is described hereunder.

In certain embodiments, an ammonia-based buffer at pH of at least 9 is used.

The use of an ammonia-based buffer, at the chosen pH, avoids introducing sodium into the animal product thus treated chemically, as would have been the case if a buffer of sodium carbonate and/or of sodium bicarbonate had been used for the chemical treatment.

Step a) of chemical treatment may for example be carried out simply by putting the animal product in contact with the ammonia solution and then impregnation of the animal product with this solution, simply by passive diffusion.

Step a) of chemical treatment may also be carried out by putting the animal product in contact with the ammonia solution and then mixing the liquid/solid mixture to promote rapid diffusion of the liquid to the center of the pieces of animal product to be treated.

Preferably, step a) of the method is carried out at room temperature, i.e. at a temperature below 45° C., for example at a temperature from 15° C. to 25° C.

### Step b): Thermal Treatment

In step b), the material resulting from the chemical treatment of the animal product, which is obtained at the end of step a), is submitted to a thermal treatment by heating in humid conditions, also called “wet heating”.

In general, it is known that a treatment of a product with wet heat gives an enhanced sterilization effect with respect to a variety of microorganisms, compared to thermal treatment with dry heat, at the same temperature.

In general, for the step of heating in humid conditions, the humidity derives from the water contained in the animal product, i.e. from the water that was contained initially in the animal product before step a) of chemical treatment and from the water derived from the aqueous ammonia composition that diffused to the center of the animal product in step a) of chemical treatment.

Preferably, step b) of thermal treatment is carried out in an atmosphere having a percentage moisture of the material to be treated of at least 80%.

“Percentage moisture” means the amount of water contained in the material to be treated. The percentage moisture of the material to be treated can easily be determined by a person skilled in the art, for example using the conventional method comprising (i) a step of weighing the material to be treated, (ii) a step of evaporation by heating of the water contained in the material to be treated, for example in a stove at 100° C. at atmospheric pressure, and then (iii) a step of weighing the material after evaporation of the water.

Step b) of thermal treatment is preferably carried out at a temperature above 70° C.

It may be below 100° C., for example in the range from 70° C. to 100° C., such as at a temperature from 75° C. to 85° C.

As is logical, the duration of step b) may vary from some minutes to some tens of minutes, in particular according to the conditions of temperature, and if applicable of pressure, of the heating step, as well as the amount of material to be treated. For example, step b) may have a duration in the range from 5 minutes to 60 minutes.

Conversely, shorter treatment times will be compensated by temperatures in the range from 100° C. to 118° C., preferably from 110° C. to 115° C.

In step b) of thermal treatment, the ammonia contained in the material to be treated evaporates in the form of ammonia gas, which is then preferably recovered in the form of ammonia by being brought into contact with water, for example with a water curtain that is interposed in the gas stream. Preferably, cooling is with water, which allows the ammonia to remain trapped in the stream of water.

The aqueous ammonia composition that is thus generated is advantageously recovered, to be used in a repetition of step a) of chemical treatment for an animal product which must subsequently be treated by the method according to the present description. The ammonia that is thus collected by dissolution in a stream of cold water is then returned to the reactor used for the chemical treatment.

The duration of step b) can easily be adapted by a person skilled in the art, based on general knowledge, in particular according to the values of temperature, of moisture, and if applicable of pressure, that were selected.

The fact that the step of thermal treatment is carried out in a humid atmosphere gives a much greater reduction of various pathogens, including viral, bacterial, and fungal pathogens. Moreover, the applicant has shown that this step of thermal treatment by “wet heating” greatly reduces the presence of unconventional pathogens, such as the presence of pathogenic proteins of the prion type. The reduction in the presence of pathogens, in particular the reduction in the presence of unconventional transmissible pathogens such as pathogenic prion protein, is further improved in the presence of citrate, the ammonia/citrate mixture being particularly effective for destroying the pathogenic proteins of the prion type.

Thus, the operating conditions of temperature and humidity in step b) of thermal treatment allow production of a material, treated chemically in step a), and then treated thermally in step b), that is substantially free, or even completely free, of transmissible pathogens. Thus, the material obtained at the end of step b) comprises mineral elements and organic elements that may be used subsequently, for example as useful inputs in agricultural practice.

### Step c): Separation of the Organic Matter and Mineral Matter

In step c), the organic matter and the mineral matter contained in the material obtained at the end of step b) are separated.

More precisely, for the material obtained at the end of step b), the organic matter and the mineral matter are collected separately, by any technique known by a person skilled in the art, for example at the level of the discharge grate of an extruder.

In certain embodiments of the method, all of the steps a), b) and c) may be carried out in a single industrial processing device. For example, in these embodiments, an extrusion device may be used, preferably a screw extruder, which includes a twin-screw extruder.

In the embodiments where an extrusion device is used for successively carrying out each of the steps a), b) and c):
for step a), the appropriate amounts of animal product and of aqueous ammonia composition are introduced, together or separately, at the level of the feed devices of the extruder, then the liquid/solid mixture obtained is mixed within the extruder, for example simultaneously with progression of this mixture along a mixing chamber of the extruder,
for step b), the material treated chemically in step a) is conveyed to a heating chamber provided in the extruder, and then the material is heated simultaneously with its progression in the heating chamber of the extruder, and
for step c), the material treated chemically, and then thermally, is conveyed to the discharge orifice of the extruder where the liquid and the solid are separated, for example at the level of the discharge grate of the extruder. In certain embodiments of step c), the material is cooled and then is brought into contact with a suitable amount of water before final pressing at the level of the discharge grate of the extrusion device.

In these embodiments, the duration of each of steps a) and b) is easily controlled, for example (i) according to the length of each of the chambers of chemical treatment and of thermal treatment and (ii) according to the speed selected for advance of the material in the course of treatment in each of the aforementioned chambers.

The organic matter was contained mainly in the solid fraction of the material obtained at the end of step b) and therefore ends up in the solid fraction separated at extruder outlet.

The mineral matter was contained mainly in the liquid fraction of the material obtained at the end of step b) and therefore ends up in the liquid fraction separated at extruder outlet.

Owing to their substantially sterile, or even completely sterile, character, substantially free from transmissible pathogens, or even completely free from transmissible pathogens, each of the (i) liquid mineral matter and (ii) solid organic matter obtained separately at the end of step c) may be used subsequently as useful input material in agriculture.

Typically, the liquid mineral matter spread in the fields corresponds to a liquid flash fertilizer leachable by rain leading to pollution of the water table and the phenomena of green algae by eutrophication of waterways. It is precisely this phenomenon of green algae that is taken advantage of in the method according to the present description and that is controlled owing to the presence of the tank in which the protein-rich plant or bacterium is cultured, in particular the microalgae or the cyanobacteria, and quite especially Spirulina.

With regard to the solid matter, in traditional agriculture, the latter corresponds to the manure to be spread and not to the feed for animals, in this case insects at the larval stage (after predigestion by the mycelium of edible lignivorous fungi which once again, in traditional agriculture would be used very differently, i.e. for their fructifications and not at all for their mycelium.

### Steps c1) and c2): Pretreatment of the Liquid and Solid Fractions

The liquid fraction and the solid fraction that are obtained separately at the end of step c) are, as will be explained in more detail later in the present description, used for their respective physicochemical and nutritional benefits. However, before their subsequent use, the liquid fraction and the solid fraction are each submitted respectively to a pretreatment, each of these pretreatments being detailed below.

### Step c1): Pretreatment of the Liquid Fraction

In step c1), the liquid fraction obtained at the end of step c) is brought into contact with a ligneous substrate colonized by an edible lignivorous fungus, and more precisely by a ligneous substrate colonized by mycelium of an edible lignivorous fungus.

The ligneous substrate consists advantageously of sawdust, wood chips or granules.

For carrying out step c1), the substrate material, for example wood chips or granules, is inoculated with an edible lignivorous fungus in its primary mycelium form, which may be selected in particular from Pleurotus ostreatus, Pleurotus pulmonarius, elm oyster mushroom (Hypsizygus ulmarius), or Agaricus blazei and Agaricus brasiliensis. Preferably, it is Pleurotus ostreatus.

During contact with the wood colonized by mycelium of an edible lignivorous fungus, the heavy metals and other potentially toxic compounds that may be contained in this liquid fraction are fixed by this colonized substrate, these undesirable compounds, if present, then being removed from said liquid fraction.

The liquid fraction from which these undesirable compounds have been removed may then be used in step d) of the method.

### Step c2) of the Method

The solid fraction obtained at the end of step c) of the method advantageously itself also undergoes a step of pretreatment, which increases its capacity for then being used as a nutrient input for insect larvae, for generating products of high added value such as proteins and oil, the residues resulting from digestion then being usable as compost.

Thus, in certain advantageous embodiments of the method, step c) is followed by a step c2) of digestion of the solid fraction obtained in step b) by putting said material (i) in contact with at least one edible lignivorous fungus, and then if applicable (ii) with insect larvae, preferably insect larvae of the species Hermetia illucens, also called “soldier fly”.

The production of compost by digestion of organic waste by insect larvae of the species Hermetia illucens is known per se and forms part of the general knowledge of a person skilled in the art.

For digestion of the solid fraction obtained in step c) by an edible lignivorous fungus, in combination with digestion by insect larvae, said material is inoculated with an edible lignivorous fungus in its primary mycelium form, which may in particular be selected from Pleurotus ostreatus, Pleurotus pulmonarius, elm oyster mushroom (Hypsizygus ulmarius), or Agaricus blazei and Agaricus brasiliensis. Preferably, it is Pleurotus ostreatus.

According to one embodiment and in order to facilitate starting of the first fermentation, the edible lignivorous fungus is precultured on a suitable culture medium before being seeded on said material. The conditions for carrying out this preculture are known by a person skilled in the art. Preculture may be carried out for example on wheat, spent brewery grains, rice or else a mixture of rice, straw and/or wood.

In certain preferred embodiments of step c2), the material obtained in step c) is inoculated with between 10% and 20% (dry weight), preferably of the order of 20% (dry weight) of the preculture of the edible lignivorous fungus and is then maintained at an optimal temperature for growth of the edible lignivorous fungus used. For example, the culture temperature is between 15° C. and 30° C., preferably of the order of 25° C.

In step c2), digestion by an edible lignivorous fungus is used when the material to be treated consists of the litter of farm animals comprising animal waste.

In step c2), first the material to be treated is inoculated with the edible lignivorous fungus. The step of colonization and digestion of the material by the edible lignivorous fungus typically takes about 1 to 5 weeks. In this step, in certain embodiments miscanthus is also added to the material to be treated.

The first step is therefore complete colonization by the mycelium of the lignivorous fungus (between 1 and 5 weeks, preferably 10 days in certain favorable conditions followed by thermal inactivation (typically at 70° C.)). The second step is bringing the substrate into contact with a suitable quantity of young larvae of Hermetia so that after a week all the substrate has been composted by the larvae, which will have reached maturity ready to be harvested (growth by a factor of 500).

The edible lignivorous fungus digests the biological polymers into units of smaller size, such as monomers, which are then absorbed by the mycelium. In this case they are the main actors in the decomposition of cellulose and lignin present in animal litter, or of keratin present in the feathers of birds, including poultry such as hens and chicken. The products of digestion by the edible lignivorous fungus are edible for humans and/or animals.

The step of digestion by insect larvae is carried out regularly, including when the material to be treated contains animal meat, or consists of animal meat.

The insect larvae and the edible lignivorous fungus can then be killed by thermal treatment.

Growth of the mycelium is stopped by moderate thermal treatment.

The insect larvae that have multiplied during the digestion step may subsequently constitute a biomass that may be transformed and may contribute to the manufacture of a feed composition for animals.

The compost resulting from digestion of the material obtained in step b) by the combination of the mycelium of edible lignivorous fungus followed by insect larvae may advantageously be used as fertilizer for the growing of plants.

It is possible to utilize the substrates so treated in many branches of industry, provided there are changes in regulations in certain countries, typically in Europe.

### Step d): Use of the Mineral Liquid Fraction

The liquid fraction that is obtained separately at the end of step c), or better still at the end of step c1), mainly contains mineral matter, combined with water-soluble organic matter also derived from the material treated chemically and then thermally, such as for example amino acids and sugars. Moreover, in particular owing to the presence of ammonia, this liquid fraction is alkaline.

In step d), this liquid fraction is added to a tank in which a protein-rich plant or bacterium is cultured, preferably protein-rich microalgae or cyanobacteria.

As will be explained in more detail hereunder, the tank used in step d) of the method
is contained in an enclosure substantially impervious to the liquid and gaseous fluids in the surroundings, said enclosure comprising a roof at least partially transparent to daylight, said roof being equipped with a plurality of photovoltaic cells, preferably Grätzel photovoltaic cells, and/or
is equipped with a device for stirring the water and recovering the protein-rich plant or bacterium,
is supplied with luminous energy, and/or
is equipped with a device regulating the temperature of the water contained in the tank.

The various nutrients, mainly mineral, but also organic, contained in the liquid fraction obtained at the end of step c) constitute a nutritional supply contributing to the growth of said protein-rich plant or bacterium, and if applicable constitute the only nutritional supply allowing growth of said protein-rich plant or bacterium. Moreover, owing to its alkaline nature, addition of this liquid fraction makes it possible to alkalize the aqueous medium in which the protein-rich plant or bacterium is growing, which promotes its growth, especially when said protein-rich plant or bacterium is a microalga, and quite especially a microalga of the genus Arthrospira, such as Spirulina. At the same time, contamination by undesirable plants, such as toxic cyanobacteria, is avoided.

Preferably, the protein-rich cyanobacteria that are cultured in said tank are of the genus Arthrospira. Preferably cyanobacteria of the species selected from Arthrospira platensis and Arthrospira maxima are used. According to a preferred choice, the protein-rich plant or bacterium is a Spirulina.

In step d), the tank that is fed with the liquid fraction obtained in step c) is temperature-controlled, the temperature of the water contained in the tank being maintained in a controlled manner at a suitable setpoint temperature.

“Suitable setpoint temperature” means a temperature allowing optimal conditions for growth of the microalgae or cyanobacteria that are cultured in the tank, this temperature forming part of the general knowledge of a person skilled in the art.

As a reminder, growth of the microalgae and cyanobacteria takes place during the times when they are exposed to daylight During the period of time when these protein-rich plants are exposed to daylight, their growth is promoted when the temperature of the water varies from 25° C. to 35° C., for example from 28° C. to 35° C.

In all cases, the temperature of the water in the tank must not exceed 43° C.

However, during the periods of time when these protein-rich plants or bacteria are not exposed to light, a lower tank temperature may be acceptable, provided that this lower temperature does not affect the survival of the protein-rich plant or bacterium, for example the cyanobacteria or the microalgae in question. Very preferably, the temperature of the water in the tank must be at least 20° C.

As detailed elsewhere in the present description, maintenance of the temperature of the water in the tank at the selected setpoint temperature may be provided by controlled supply of calories or frigories (negative calories), as needed, which are generated within a system, called here system for treating animal products, of which said tank is one of the constituent elements, said system being designed to operate optimal recycling of the flows of energy and of chemical elements, for (i) optimal utilization of the energy and of the chemical elements generated by application of the method in said system and thus (ii) drastically reduce (ii-a) the need for energy and chemical inputs and (ii-b) the production of waste that cannot be reused.

Thus, in certain embodiments of step d) of the method, the tank to which the liquid fraction that was obtained at the end of step c) is added comprises a plurality of technical characteristics contributing to the conservation of energy and of the organic and mineral elements, which may be used by other devices making up a system to which the tank used in step d) belongs.

Preferably, the tank is of circular or ovoid shape.

In certain embodiments, the tank is contained in a closed enclosure substantially impervious to exchanges of liquid and gaseous fluids.

Said enclosure containing the tank comprises a roof. Said roof consists, preferably on the whole of its surface, of a covering material substantially transparent to light, said roof being equipped with a plurality of photovoltaic cells, said photovoltaic cells preferably being Grätzel cells.

The presence of the roof contributes to isolation of the aquatic and atmospheric contents of the enclosure in which the tank is contained, from the surroundings and thus allows control in the selected manner of the exchanges of energy, and of gaseous and liquid fluids, or solids between the sealed enclosure and the surroundings, and also protects against external contamination, for example by algae, bacteria or viruses.

Preferably, the roof of the enclosure containing the tank consists, at least on a part of its surface, and if applicable on the whole of its surface, of a material at least partially transparent to daylight. It may be a covering element made of glass, or of a polymer material transparent to daylight, such as natural glasses or synthetic glasses, in particular glasses made of polymer, or Plexiglas. Preferably, the covering element is made of a material having a transparency to daylight of at least 50%, better still at least 80%, even better at least 90%, relative to the transparency of a glass screen at least 6 mm thick.

Preferably, the roof is equipped with a plurality of photovoltaic cells. Said photovoltaic cells, when they are exposed to daylight, generate electrical energy, which may be used extemporaneously and/or be stored with a view to subsequent use thereof, for example for actuating other elements or devices making up the system for treating animal products, which is described in detail later in the present description.

With the system according to the present description, battery storage of the electrical energy that has been produced but not consumed is not favored, in particular on account of the environmental and financial cost of existing devices for storage of electrical energy.

With the system according to the present description, the power of the elements that generate electrical energy, quite especially the photovoltaic cells, will be determined so as to be in balance with the demand for electrical energy of all of the other elements of said system.

However, it may nevertheless happen that a small part of the electrical energy produced by the system according to the present description is not used. In this case, the surplus electrical energy may be distributed temporarily on the basis of a device of the “smart grid” type, well known in the prior art.

Preferably, photovoltaic cells are used whose presence does not substantially alter the passage of daylight through the transparent, or partially transparent, roof and therefore does not substantially reduce the exposure to daylight of the protein-rich plant or bacterium cultured in the water in the tank. The reduction in the transmission of daylight caused by the presence of the photovoltaic cells makes it possible to avoid the protein-rich plant or bacterium being exposed to an excessive amount of light, which is a source of photo-inactivation, likely to affect their survival in the tank.

Preferably, the photovoltaic cells selected are so-called Grätzel cells, which are familiar to a person skilled in the art. As is known, in Grätzel photovoltaic cells, photon absorption and charge transport are separated in the dye solar cell. A Grätzel cell consists of a cathode and an anode, made of conductive glass, on which there is a layer of titanium dioxide (TiO₂), which is a semiconductor, on the surface of which a sensitizer or dye is absorbed, an aqueous solution having the function of electrolyte being enclosed between the two plates delimiting the photovoltaic cell. Grätzel photovoltaic cells have the advantage of letting through at least 50% of the light, which is why these photovoltaic cells are sometimes called “transparent cells”.

The number of photovoltaic cells arranged on the surface of the roof of the enclosure containing the tank can easily be determined by a person skilled in the art, as a function of the area of said roof and if applicable depending on the amount of electrical energy required for proper operation of the system for treating animal products. In certain embodiments of the system, photovoltaic cells are arranged on the whole surface of the roof.

As already mentioned above, absorption of a part of the daylight by their interposition between the light and the protein-rich plant or bacterium cultured in the tank is not a drawback. On the contrary, in many situations, more moderate exposure of the plant or bacterium to sunlight is favorable for growth of this plant or of this bacterium, for example is favorable for the growth of certain cyanobacteria or microalgae such as Spirulina.

In certain embodiments, the tank is equipped with a device for stirring the water and recovering the protein-rich plant or bacterium, for example Spirulina.

In particular, in the embodiments in which the tank is circular, or if applicable of ovoid shape, the device for stirring the water may be a rotary device comprising a vertical shaft defining a rotation axis, which is preferably located at the center of the tank, said device comprising at least one arm, perpendicular to the rotation axis and fixed on the vertical shaft, movement of the arm being actuated by the rotation of the shaft, said arm preferably being fixed on the shaft at a height corresponding to the water/air interface of the water contained in said tank. Thus, when the arm is rotating, it causes, by its submerged surface, turbulence generating mixing of the water in the tank, if applicable contributing to oxygenation of the water in the tank. Moreover, the portion of surface of the rotating arm that is at the water/air interface makes possible, by scraping the upper part of the water in the tank, the regular recovery of a part of the mass of the protein-rich plant or bacterium growing in said tank, for example Spirulina.

In certain embodiments, the collecting arms are of curved shape, so that the harvested plants move, owing to the water current generated by the rotation of the arms, from the periphery of the arm to the central axis where the central shaft is located. Moreover, the central shaft preferably comprises an endless screw (Archimedes screw), toward which the plants moving from the periphery to the axis of the device are directed. Thus, after moving horizontally, the harvested plants move vertically along the central shaft, and are then recovered at the outlet of the endless screw.

In certain embodiments of the system according to the present description, in particular in the embodiments in which a tank of very large diameter, for example with a diameter of more than 100 meters, is used, the device for mixing and recovery comprises a plurality of arms fixed on the central shaft, the horizontal axis of a given arm making a defined angle with the horizontal axis of the preceding or following arm, the plurality of arms together covering the whole of the central shaft, or 360 degrees. As an illustration, for an embodiment of the mixing device for which 10 horizontal arms are fixed on the central shaft, a given arm is preferably oriented at an angle of 36 degrees, both relative to the preceding arm and relative to the following arm.

In certain embodiments, the depth of water in the tank is low, of the order of 20 cm to 50 cm, with the result that the exposure to daylight of the protein-rich plant is sufficient to allow its growth.

In other embodiments of the tank, the depth of water is greater than 50 cm. It may for example be up to a depth of water of 3 meters. In these embodiments, the conditions for good growth of the protein-rich plant or bacterium are not met, owing to insufficient exposure of this plant or of this bacterium to daylight, in the deep part of the tank.

In certain embodiments of the mixing device, rotation of the latter is provided by a motor coupled to the central shaft.

In other embodiments of the mixing device, in particular in the embodiments in which the mixing device is dimensioned for equipping a tank of large diameter, for example a tank with a diameter of 100 meters or more, preference will be given to a means of rotation in the form of a plurality of motors equipping the end of a plurality of arms making up the mixing device, therefore located at the periphery of the tank.

In certain embodiments, the tank may have a depth of water that is not compatible with growth of the protein-rich plant or bacterium for the entire depth of water in the tank, for example because an excessive depth does not allow sufficient exposure of the protein-rich plant or bacterium to daylight. In these embodiments of the tank, the insufficiency of access of the plant or bacterium to daylight is compensated by the presence on the walls of the submerged part of the tank and/or on the walls of the rotary arm or arms, of a plurality of electroluminescent sources, for example LEDs, able to supply luminous energy to the protein-rich plant or bacterium, sufficient for its growth.

In these embodiments, the device for mixing and recovery may be equipped with a plurality of arms perpendicular to the rotation axis of the shaft on which they are fixed, including a first arm for mixing and recovery, perpendicular to the rotation axis, fixed on the shaft at a height located at the level of the water/air interface, and at least one other arm perpendicular to the rotation axis fixed on the shaft at a height such that said arms are completely submerged in the water in the tank and are each equipped with a plurality of electroluminescent sources able to emit light at at least one wavelength promoting growth of the protein-rich plant or bacterium, for example in the near ultraviolet.

According to other embodiments, the tank, in which the liquid fraction is added in step d) of the method, is temperature-controlled. According to these other embodiments, the tank is equipped with a temperature regulating device for heating or cooling the water in the tank. The device is regulated by a monitoring/control system allowing the temperature of the water contained in the tank to be maintained at the selected setpoint temperature.

In general, in a system according to the present description, the water contained in the tank constitutes a mass supplying thermal inertia; this thermal inertia mass may contribute to counterbalancing the production of heat by other modules of the system, when present, such as a bioclimatic greenhouse unit, or a plurality of bioclimatic greenhouse units.

For heating the water in the tank, the temperature regulating device preferably comprises a heat exchanger system that is in fluidic communication with the hot water outlet pipe of an apparatus for desalination of the water by evaporation/concentration Heat may also be supplied to the tank by the calories that may be supplied by the bioclimatic greenhouse unit or units forming part of the system according to the present description. The devices for water desalination by evaporation/concentration are well known in the prior art. This type of device is described for example in the certificate of addition to a patent of invention No. 95,887 filed on 8 Nov. 1968 in the name of the Commissariat à l'Energie Atomique (Atomic Energy Commission) and granted on 4 Oct. 1971.

For cooling the water in the tank in summer, the temperature regulating device comprises a heat exchanger system of the geothermal type.

In certain embodiments, the bottom of the tank is equipped with a metal plate that will give considerable heat exchange with the water in the tank, in order to cool the water in the tank.

This plate normally serves for withdrawing frigories (negative calories), and therefore supplying calories (typically heating to 45° C.).

This makes it possible to distribute with a cooled heat transfer fluid.

If the temperature of the tank tends to rise too much, it is advantageous to have the possibility of reversing the system and using the bottom plate for withdrawing calories from the tank, transferring them to the subsoil.

The heat exchanger system of the geothermal type, preferably of a known type, comprises a pipe, or a plurality of pipes, in which a heat transfer fluid circulates, for example water, the pipe or pipes being located at a selected depth below the surface of the ground, said fluid being cooled before being returned to the heat exchanger proper, for the purpose of controlled cooling of the water in the tank.

According to yet other embodiments, the tank may be supplied with water, for example to compensate a loss of volume owing to evaporation.

According to these other embodiments, the tank may be provided with a controllable device for water supply, said water supply device being in fluidic communication with a fresh water outlet pipe of a device for desalination of water by evaporation/concentration.

The desalination device by evaporation/concentration offers the advantage of fixing calcium and other insoluble salts on the polymer membranes contained therein. Thus, the desalinated water feeding the tank is substantially free from, or completely free from, calcium salts or other insoluble salts, which are undesirable because they would precipitate in an alkaline medium and would affect the growth of the protein-rich plant or bacterium, in particular the microalgae or the cyanobacteria, such as Spirulina.

According to yet other embodiments, the enclosure of the tank may be supplied with carbon dioxide in the form of carbonate and with nitrogen in the form of ammonia, which are fed into the atmosphere of the enclosure. The carbon dioxide and the nitrogen supplied to the atmosphere of the enclosure may consist of gases that are produced in steps c1) and c2) of digestion of the material obtained in step c) by the combination of insect larvae and the mycelium of edible lignivorous fungi.

According to yet other advantageous embodiments, calories derived from the bioclimatic greenhouse unit(s) making up the system, when they are present, are supplied to the desalination device by evaporation/concentration.

### System According to the Present Description

The present description also relates to a system for treating animal products comprising a plurality of treatment units, said system being designed for constituting a substantially self-sufficient system, i.e. to constitute a system which, after it has been started, requires reduced, or no, external supplies of energy and materials. The implementation of the system according to the present description, if it is operated optimally, may even lead to a negative carbon balance.

According to this design, operation of the system for treating animal products according to the present description is environmentally friendly and is compatible with the pursuit of sustainable development. Moreover, owing to the reduced external supplies of energy and materials required for its operation, the system for treating animal products according to the present description can be installed in an environment with reduced resources, and quite particularly reduced water resources. Also, owing to the possibility of implementing optimal reuse of the various materials produced, operation of the treatment system generates a reduced amount of waste that cannot be utilized. Thus, operation of the treatment system according to the present description supplies a variety of organic matter and mineral matter useful for human and animal nutrition.

The present description relates to a system for treating animal products, said system comprising:
a reactor for chemical and thermal treatment of said animal products,
an extrusion device equipped with a controlled heating system, which may be in fluidic communication with said reactor, the outlet of the extrusion device being equipped with a liquid/solid separator,
a closed enclosure comprising a tank surmounted by a roof consisting of a material substantially transparent to light, said tank being fillable with water and intended for culture of a plant or a bacterium, in particular a protein-rich plant or bacterium such as protein-rich microalgae or protein-rich cyanobacteria,
a system for water desalination by evaporation/concentration comprising (i) a salt water supply pipe, a pipe for discharge of the water that has not been desalinated and an outlet pipe for desalinated water, it being specified that:
optionally, said reactor is in fluidic communication with the extrusion device,
said tank is provided with a controllable device for water supply, said water supply device being in fluidic communication with a salt water outlet pipe from the desalination system.

The reactor for chemical and thermal treatment of animal products comprises a treatment enclosure and at least one means for supplying said reactor with an ammonia-based buffer and a means for heating the animal products in said reactor. In certain embodiments, said reactor is an extruder comprising a means for supplying animal products and a means for supplying ammonia-based buffer.

The heating means may be of any known type. In certain embodiments said reactor is equipped with a heating device by heat exchange, for example such as a plate exchanger device of a known type, in which a hot fluid circulates. In certain embodiments, the hot fluid circulating in the heat exchanger is hot water derived from a device for desalination of water by evaporation/concentration.

The liquid/solid separator is equipped respectively with an outlet pipe for the solids and an outlet pipe for the liquid.

In certain embodiments, the outlet pipe for the liquid is in fluidic communication with the tank contained in the closed enclosure. Thus, the liquid fraction at reactor outlet, for example from the extruder, is used as feed to the tank contained in the closed enclosure.

In certain embodiments, the tank contained in the closed enclosure is equipped with a temperature regulating device for heating and cooling the tank, which is described in detail elsewhere in the present description.

In certain embodiments, the desalination system comprises an outlet pipe for salt water and an outlet pipe for desalinated water.

In certain embodiments, the outlet pipe for desalinated water is in fluidic communication with the tank contained in said closed enclosure, for the purpose of supplying said tank with salt water.

In certain embodiments of the system, the latter further comprises a bioclimatic greenhouse unit. The bioclimatic greenhouse unit may be, in general respects, of a type known in the prior art, except for the technical characteristic or characteristics specified below, which make said bioclimatic greenhouse unit suitable for being integrated as an element of the system according to the present description.

In certain embodiments, said system further comprises a bioclimatic greenhouse device comprising:
a closed enclosure comprising a floor and a roof, the floor being located below ground level,
said roof being substantially transparent to light, on the wall of which there is a plurality of photovoltaic cells, preferably Grätzel cells,
said enclosure being provided with a device for dehumidifying the internal atmosphere of the enclosure,
said enclosure being temperature-controlled,
a substrate suitable for growing plants being disposed on the surface of said floor, said substrate consisting, at least partly, of the solid fraction of the product from animal waste treatment, in, successively, the reactor and then the extrusion device.

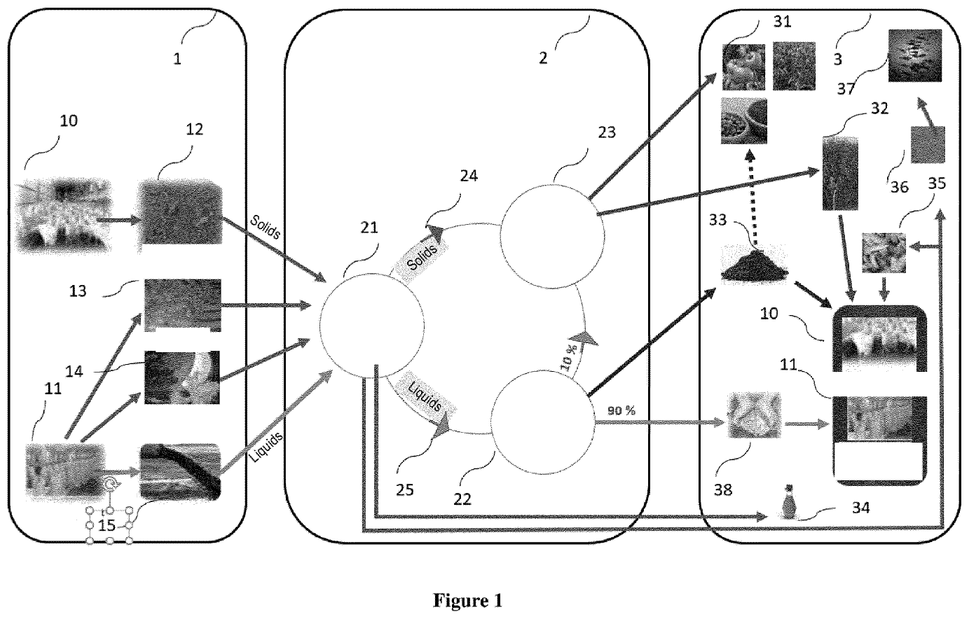

A general schematic of a treatment system according to the present description is shown in FIG. 1. The schematic in FIG. 1 shows three boxes, respectively on the left, in the center and on the right of the FIGURE.

The box on the left 1 relates to the animal farming unit 10 and shows the various animal products that are generated by farming, received from the farm, either directly, or after treatment in a processing unit 11, such as dung 12, animal offal 13, poultry feathers 14 and wastewater 15.

The central box 2 shows schematically the three main treatment units, respectively:
the chemical and thermal treatment unit 21 of the animal starting products, where said unit may, in certain embodiments of the system, comprise a unit for treatment of products, previously treated chemically and thermally, by digestion by a combination of larvae of Hermetia illucens and at least one edible lignivorous fungus,
the closed enclosure in which there is a tank 22 able to allow growth of a protein-rich plant or bacterium,
the bioclimatic greenhouse unit 23.

The box on the right 3 shows schematically the various flows of materials that are generated by operating the system for treating animal products. These various materials may be used for human nutrition, for animal nutrition, in particular as feed for the animal farm forming part of the system, and for other aspects of animal farming practice, for example as litter for the animals.

The box on the left 1 shows the various aspects associated with the animal farming unit. The part top left shows the farming unit 10. Operation of the farming unit generates waste produced by the animals, mainly products consisting of their droppings 12. combined with the mixture of materials making up their litter. The farming unit also generates carbon dioxide, which may be recovered and fed back (i) into the enclosure containing the tank 22 for culture of a protein-rich plant or bacterium and/or (ii) into the bioclimatic greenhouse unit 23, in which carbon dioxide may be absorbed by the plants cultivated in the greenhouse and may thus contribute to the growth of these plants.

The lower part of the box on the left 1 shows the processing and production unit 11 of the animal products, which are mainly intended as human food. Operation of the processing unit 11 generates a variety of animal products that are not used for human nutrition, which includes (i) offal 13, (ii) wastewater 15, which is generated by the various steps of treatment of dead animals in the processing unit and (iii) if applicable, when the farm animals are birds kept for profit, in particular poultry such as geese, turkeys, ducks, hens and chicken, guinea fowl, capons, quails, pheasants and pigeons, also the feathers 14 of these animals.

As a reminder, the central box 2 shows schematically (i) the chemical and thermal treatment unit 21, (ii) the closed enclosure comprising the tank 22 suitable for culture of a protein-rich plant or bacterium and (iii) the bioclimatic greenhouse unit 23 On the left part of the central box 2, it is shown that the chemical and thermal treatment unit 21 is supplied respectively with (i) the solid waste 12 generated by operation of the farming unit, (ii) the solids consisting of the offal 13 derived from operation of the processing unit, (iii) if applicable, the solids consisting of feathers 14 of the farm animals and (iv) the liquids mainly consisting of the wastewater 15 generated by operation of the processing unit 11. As is described elsewhere in the present description, these solid and liquid materials are treated thermally and chemically in steps a) and b) of the method according to the present description. If applicable, the material that has been treated chemically and thermally is then submitted to a step b1) of digestion by a combination of insect larvae and mycelium of edible lignivorous fungi. As has been described above, the material obtained at the end of step b) of the method, if applicable at the end of step b1) of digestion, is generated in the form of a material comprising a solid fraction 24 and a liquid fraction 25, which are separated in step d) of the method.

The solid fraction 24 is used for supplying nutrients, useful for plant growth, to the bioclimatic greenhouse unit 23, shown at top right of the central box 2 in FIG. 1.

The liquid fraction 25, which is an alkaline liquid comprising mineral elements and organic elements, is used for supplying nutrients, useful for growth of the protein-rich plant or bacterium, for example a plant or a bacterium of the genus Arthrospira such as Spirulina, to the closed enclosure unit comprising the tank 22 in which the protein-rich plant or bacterium is growing.

Moreover, the larvae resulting from multiplication of the insect larvae during step b1) of the method, in the chemical and thermal treatment unit 21, may be used as a source of proteins for purposes of manufacture of food compositions intended for (i) human nutrition and/or (ii) animal nutrition, and preferably for feeding the farm animals within the farming unit of the system according to the present description.

The box on the right 3 in FIG. 1 shows schematically the various products that are generated by each of the units (i) of chemical and thermal treatment, (ii) the bioclimatic greenhouse unit and (iii) the closed enclosure unit comprising the tank in which the protein-rich plant or bacterium is growing.

As can be seen on the central box 2 in FIG. 1, the bioclimatic greenhouse unit 23 is used for growing food plants, vegetables and/or fruits 31, mainly intended for human food. If applicable, certain parts of the plant, not used for human nutrition, may be used (i) for feeding the animals in a farm, or (ii) for supply a material making up the litter for the animals 32.

As can also be seen in the schematic in FIG. 1, operation of the tank unit 22 generates a vegetable or bacterial mass consisting of the protein-rich plant or bacterium 33 that has been collected, for example in real time as they grow; said protein-rich plant or bacterium may be used as a constituent for human nutrition 31 or animal nutrition 31, for example for feeding the animals bred in the farming unit 10 of the system according to the present description. Moreover, the water contained in the tank 22, which has been filtered and purified by the protein-rich plant or bacterium, may then be supplied (i) to the farming unit 10 and/or (ii) to the processing unit 11, as it is useful for the proper operation of each of these units of the system according to the present description.

As can also be seen in the schematic in FIG. 1, the chemical and thermal treatment unit 21 may generate a lipid-rich fraction 34.

As can also be seen in the schematic in FIG. 1, the chemical and thermal treatment unit 21 may generate a solid fraction, processing of which may be provided by insect larvae 35, and the resultant processed material may subsequently be used in particular for feeding animals within the farm 10 (see box on the right 3).

As is also shown in the schematic in FIG. 1, unsalted water 38, in particular pure water, may be taken from tank 22 to feed the processing unit 11.

Moreover, the insects 36 from the insect larvae may be used as protein input, for example in an aquaculture unit 37.

In certain embodiments of the system, said tank 22:
is a tank that is covered, preferably over the whole of its upper surface, with a covering element transparent to light equipped with a plurality of photovoltaic cells, said photovoltaic cells preferably being Grätzel cells, and/or
is equipped with a device for stirring the water and recovering Spirulina, and/or
is supplied with luminous energy, (i) on the one hand, by natural light transmitted by the covering element and (ii) on the other hand, by light emitted by a plurality of electroluminescent sources arranged on the walls of the tank and/or on the walls of the device for mixing the water and recovering Spirulina, and/or
is equipped with a heat exchanger device for heating and/or cooling the water, said device being regulated by a monitoring/control system for maintaining the temperature of the water contained in said tank at the setpoint temperature.

In certain embodiments of said system, the temperature regulating device of the enclosure comprises a heat exchanger in fluidic communication with the water desalination system.

In certain embodiments, said system further comprises an agroforestry arrangement comprising:
an area planted with shade-generating trees arranged in rows suitably spaced to provide a tunnel greenhouse, or a plurality of tunnel greenhouses, between two rows of said trees,
a tunnel greenhouse, or a plurality of tunnel greenhouses, arranged between two rows of trees, and
a system for irrigation of the trees, and if applicable a system for irrigation of the plants that may be cultivated in the tunnel greenhouse(s), said irrigation system being in fluidic communication with the desalinated water outlet of the system for water desalination by evaporation/condensation.

The present description also relates to a method for decontaminating animal products to remove pathogens, comprising the following steps:
a) chemical treatment by putting the collected waste in contact with an ammonia-based buffer at pH of at least 8, and
b) thermal treatment of the material obtained in step a), by heating said material in humid conditions at a temperature of at least 70° C.

More specific characteristics of the execution of steps a) and b) are detailed elsewhere in the present description, in connection with the description of steps a) and b) of the method for treating animal products.

The pathogens include pathogenic microorganisms, such as pathogenic bacteria and pathogenic viruses. The pathogens also include the unconventional transmissible agents (UTAs), such as pathogenic viroids and prion proteins.

In certain embodiments, the method for decontaminating animal products to remove pathogens further comprises a step c) of putting the material obtained at the end of step b), if applicable, of the solid fraction of the material obtained at the end of step c) when said material comprises a solid fraction and a liquid fraction, in contact with insect larvae, preferably insect larvae of the species Hermetia illucens.

More specific characteristics of execution of step c) are detailed elsewhere in the present description, in connection with the description of step c2) of the method for treating animal products.

### Example: Effect of the Method for Decontamination of Pathogens

As is illustrated in the Example, the method of treatment of animal products according to the present description, owing to the combination of (i) the basic pH supplied by the ammonia-based buffer in the chemical treatment step and (ii) the temperature applied in the thermal treatment step, makes it possible to produce organic matter that may subsequently be used in particular in agriculture, for example as fertilizers or as nutrients, said organic matter being free from pathogens, and in particular being free from pathogens known to be resistant to many thermal and chemical treatments, such as the pathogenic prion proteins.

As a reminder, it is the extraordinary resistance of the pathogenic prion proteins (responsible in particular for bovine spongiform encephalopathy, so-called “mad cow disease”, transmissible to humans and then responsible for the Creutzfeldt-Jakob variant of the disease) that has led hospital units to modify sterilization practice by autoclaving, changing from a temperature of 121° C. as used previously, to a temperature of 134° C. for 18 minutes. It is this resistance of the pathogenic prion proteins that explains why the earlier treatments of animal meal, which destroyed all the other pathogens, were ineffective for preventing the transmission of the pathogenic prion proteins causing the so-called “mad cow” crisis.

The capacity of the method of treatment of animal products according to the present description for decontaminating said animal products to remove pathogens, in particular owing to the application of steps a) and b) of this method, and in certain cases also owing to the application of step c2) by being brought into contact with insect larvae of the species Hermetia illucens, was tested in the conventional model used for studies of prion decontamination, namely by the use of the 263K strain in the hamster.

The principle of this test was to compare a representative sample of animal products contaminated with a pathogenic prion protein. As the representative sample of animal products contaminated with a pathogenic prion protein, we used a hamster brain homogenate infected with the prion at the end stage of the disease, which was used for contaminating a piece of meat, in a weight ratio of 10 wt % of hamster brain homogenate and 90 wt % of initially uncontaminated meat, relative to the total weight of the resultant animal product. The 10%:90% weight ratio was used in order to avoid any notable change in the physicochemical properties of the meat matrix while supplying a maximum amount of infectious agent and, in the particular case of this prion strain 263K, the infectious titer was greater than 10⁹ LD₅₀/g of hamster brain, i.e. amounts of infectious agent contained in 1 gram of infected hamster brain capable of killing more than one billion animals with a probability of 50% (definition of the 50% lethal dose).

This meat matrix contaminated with the 263K strain was carefully homogenized to generate a series of samples of identical volume and weight.

Then the samples either were, or were not, submitted to various decontamination treatments for the purpose of comparing the capacity for decontamination of the various treatments applied and thus judge their relative efficacy, relative to one another and relative to serial dilutions of untreated infectious samples (to determine the reduction in the infectious titer of the initial sample).

The final test consisted of inoculating the samples resulting from the various treatments applied, intracerebrally to determine this notorious 50% lethal dose and define the most effective decontamination treatments.

The positive controls consisted of:
1) Autoclave treatment at 134° C. for 18 minutes according to the WHO recommendations, which leads to a reduction of 2.5 logs of the infectious titer, or a reduction by a factor of about 300 times, i.e. less than is described classically on account of the effect of protection by the meat matrix.
2) A longer treatment at a lower temperature (60 min at 115° C.), easier to obtain industrially, and which would have less effect on the organoleptic qualities of the products treated, which leads to a reduction of 2 logs of the infectious titer, or a reduction by a factor of 100.

The treatments of the samples of the animal product consisted of:
1) A combination of lower temperature (60 min at 115° C.) with a controlled supply of ammonia-based buffer to raise the pH (here, the composition used was: 6% ammonia 30% v/v, ammonium citrate 25 mM, sodium citrate 250 mM; this buffer was mixed with the meat in a proportion of 1 volume/5 parts of meat, i.e. in practice 2 ml of buffer per 10 g of meat before thermal treatment) which leads to a reduction of 5 logs of the infectious titer, i.e. a factor 1000 times greater than the thermal treatment alone and 300 times greater than that recommended by the WHO.
2) A combination of the preceding treatment with biotransformation by Hermetia illucens, which gives more than 8 logs of reduction of the infectious titer, i.e. more than 300 000 times greater than the WHO reference treatment with no residual infectivity detected in spite of the massive infectious doses used for contaminating the samples treated.

These experiments, which required more than 12 months of follow-up of the animals, made it possible to demonstrate the superiority of the method for treating animal products according to the present description, compared to the conventional methods of decontamination.

### LIST OF DOCUMENTS CITED

Baba et al. 2017. Traditional methods of carcass disposal: a review. Journal of Dairy, Veterinary & Animal Research. 2017; 5(1):21-27.
Baba et al. 2018. Economics of Fermentation of Poultry Farm Waste. International Journal of Current Microbiology and Applied Sciences. ISSN: 2319-7706 Volume 7 Number 06 (2018)
Bolan et al. 2010. Uses and management of poultry litter. World's Poultry Science Journal, Volume 66, Issue 4, December 2010, pp. 673 698 DOI: https://doi.org/10.1017/S0043933910000656
Brandelli et al. 2015. Microbial enzymes for bioconversion of poultry waste into added-value products. Food Research International 73 (2015) 3-12
Pereira et al. 2019. Ammonia and greenhouse gas emissions following the application of clinoptilolite on the litter of a breeding hen house. Environ Sci Pollut Res Int. 2019 March; 26(8):8352-8357. doi: 10.1007/s11356-019-04429-2.

1. A method for treating animal products comprising the following steps:
a) chemical treatment by putting the collected waste in contact with an ammonia-based buffer at pH of at least 8,
b) thermal treatment of the material obtained in step a), by heating the material in humid conditions at a temperature of at least 70° C.,
c) separating the organic matter and the mineral matter in the presence of water from the material obtained in step b) and then separate collection (i) of the liquid fraction comprising the mineral matter and (ii) of the solid fraction comprising the organic matter, and
d) adding the liquid fraction obtained in step c (ii), comprising the mineral matter, to a tank filled with water maintained in a controlled manner at a setpoint temperature from 20° C. to 42° C. in which a protein-rich plant or bacterium is cultured.

2. The method as claimed in claim 1, the tank used in step d):
is contained in an enclosure substantially impervious to the liquid and gaseous fluids in the surroundings, the enclosure comprising a roof at least partially transparent to daylight, the roof being equipped with a plurality of photovoltaic cells, and/or
is equipped with a device for stirring the water and recovering the protein-rich plant or bacterium,
is supplied with luminous energy, and/or
is equipped with a device regulating the temperature of the water contained in the tank.

3. The method as claimed in claim 2, for heating the water contained in the tank, the temperature regulating device is in fluidic communication with an outlet pipe of the hot water from apparatus for desalination by evaporation/concentration

4. The method as claimed in claim 2, the heat exchanger, for cooling the water, comprising a geothermal exchanger.

5. The method as claimed in claim 1, the tank being provided with a controllable device for water supply, the water supply device being in fluidic communication with a salt water outlet pipe of a device for desalination of water by evaporation/concentration.

6. The method as claimed in claim 1, step c) being followed by a step c1) of putting the liquid fraction of the material obtained in step c) in contact with a ligneous substrate colonized by an edible lignivorous fungus and a step c2) of putting the solid fraction obtained in step c) (i) in contact with at least one edible lignivorous fungus, then if applicable (ii) with insect larvae.

7. A system for treating animal products, the system comprising:
a reactor for chemical and thermal treatment of the animal products,
an extrusion device equipped with a controlled heating system, which may be in fluidic communication with the reactor, the outlet of the extrusion device being equipped with a liquid/solid separator,
a closed enclosure comprising a tank surmounted by a roof consisting of a material substantially transparent to light, the tank being fillable with water and intended for culture of a plant or a bacterium,
a system for water desalination by evaporation/concentration comprising (i) a salt water supply pipe, a pipe for discharge of the water that has not been desalinated and an outlet pipe for desalinated water,
it being specified that:
optionally, the reactor is in fluidic communication with the extrusion device,
the tank being provided with a controllable device for water supply, the water supply device being in fluidic communication with a salt water outlet pipe from the desalination system.

8. The system as claimed in claim 7, further comprising a bioclimatic greenhouse device comprising:
a closed enclosure comprising a floor and a roof, the floor being located below ground level,
the roof being substantially transparent to light, on the wall of which there is a plurality of photovoltaic cells,
the enclosure being provided with a device for dehumidifying the internal atmosphere of the enclosure,
the enclosure being temperature-controlled,
a substrate suitable for growing plants being disposed on the surface of the floor, the substrate consisting, at least partly, of the solid fraction of the product from animal waste treatment in, successively, the reactor and then the extrusion device.

9. The system as claimed in claim 8, the temperature regulating device of the enclosure of the bioclimatic greenhouse device comprising a heat exchanger in fluidic communication with the water desalination system.

10. The system as claimed in claim 7, further comprising, an agroforestry arrangement comprising:
an area planted with shade-generating trees arranged in rows suitably spaced to provide a tunnel greenhouse, or a plurality of tunnel greenhouses, between two rows of the trees,
a tunnel greenhouse, or a plurality of tunnel greenhouses, arranged between two rows of trees, and
a system for irrigation of the trees, and if applicable a system for irrigation of the plants that may be cultivated in the tunnel greenhouse(s), the irrigation system being in fluidic communication with the desalinated water outlet of the system for water desalination by evaporation/condensation.
